(19) United States
(12) Patent Application Publication
(10) Pub. No.: US 2004/0258624 A1
(43) Pub. Date: Dec. 23, 2004

(54) Combined doses

(75) Inventors: Thomas Nilsson, Mariefred (SE); Mattias Myrman, Stockholm (SE); Claes Friberg, Akers Styckebruk (SE); Sven Calander, Strangnas (SE)

Correspondence Address: OBLON, SPIVAK, MCCLELLAND, MAIER & NEUSTADT, P.C., 1940 DUKE STREET, ALEXANDRIA, VA, 22314, US

(73) Assignee: Microdrug AG, Hergiswil (CH)

(21) Appl. No.: 10/603,818

(22) Filed: Jun. 26, 2003

(30) Foreign Application Priority Data
Jun. 19, 2003 (SE) 0301816-5

(51) Int. Cl. A61L 9/04; A61K 9/14; A61K 9/64

(52) U.S. Cl. 424/46

(57) ABSTRACT

The present invention discloses a method and a pharmaceutical dry powder combined dose for the prophylaxis or treatment of a respiratory disorder in a mammalian host by inhalation of a metered dry powder combined dose of finely divided dry medication powders. At least one dry powder medicament is selected from a first group of bronchodilating medicaments and at least one dry powder medicament from a second group of anti-inflammatory medicaments. A metered dry powder medicinal combined dose comprising separately metered deposits of medicinally suitable quantities of each of the selected medicaments is prepared, in which the sum of the metered deposits constitutes the metered quantities of powder of the combined dose and the medicinal combined dose is introduced into an adapted inhaler device for a generally simultaneous delivery of the medicinal combined dose during the course of a single inhalation by a user, such that the delivered medicinal combined dose is composed of a high proportion of mixed de-aggregated fine particles of the selected medicaments, whereby an desired therapeutic or treating effect to the user is achieved.

### TECHNICAL FIELD

The present invention relates to combined doses of asthma medicaments for administration by an oral inhalation route to a user in need of treatment of asthma and other respiratory disorders. In particular, combined doses are packaged to suit a new method of aerosolizing a selected combined dose into air and more particularly, the invention relates to combinations of separate dry powder formulations of different asthma medicaments constituting a combined dose intended for delivery in a single inhalation by a user.

### BACKGROUND

Administration of drugs by an oral inhalation route is very much in focus today, because of advantages offered like rapid and predictable onset of action, cost effectiveness and high level of comfort for the user. There are mainly two types of inhalers on the market, pressurized metered dose inhalers (pMDIs) comprising a suspension of fine medicament particles in a propellant gas and dry powder inhalers (DPIs) comprising fine medicament particles as dry powder.

Dry powder inhalers (DPI) attract perhaps the most interest, compared to pMDIs, because of the flexibility they offer in terms of nominal dose range i.e. the amount of active substance that can be administered in a single inhalation. So far most development efforts have been directed towards producing effective drugs and formulations for specific abnormal conditions and not so much towards developing combined dose metering and the delivery devices i.e. the inhaler.

When inhaling a combined dose of dry medication powder it is important to obtain by mass a high fine particle fraction (FPF) of particles with an aerodynamic size preferably less than 5 μm in the inspiration air. The majority of larger particles does not follow the stream of air into the many bifurcations of the airways, but get stuck in the throat and upper airways. It is not uncommon for prior art inhalers to have an efficacy of 10-20% only, i.e. only 10-20% of the metered dose by mass is actually delivered as particles with an aerodynamic size less than 5 μm. Since most drugs may have undesirable side effects, e.g. steroids delivered to the system, it is important to keep the dosage to the user as exact as possible and to design the delivery system, e.g. an inhaler, such that the efficacy becomes much higher than 10-20%, thereby reducing the required amount of drug in the dose. Also, depending on the targeted site of action in the airways and lungs, it is desirable to tailor the physical formulation of a medication powder in such a way that it results in an advantageous particle aerodynamic size distribution by mass in the metered dose.

Interestingly, in the past decade research into respiratory diseases, their prophylaxis and treatment, has shown conclusively that simultaneous administration of combinations of different medicaments may improve the clinical condition of patients considerably. This comes as no surprise to a person of ordinary skill in the art, since it is well known in prior art that a succesful treatment of a medical condition may require administration of more than one active substance, e.g. a medicament for relaxing the immediate symptoms like bronchoconstriction and another medicament for treating the underlying airway inflammation. In Switzerland patients diagnosed with asthma have been prescribed FORADIL (formoterol, a bronchodilating substance) together with PULMICORT (budesonide, an anti-inflammatory steroid) since the 1980's for treatment of their asthma. Until recently, however, different asthma medicaments have generally been administered separately, in sequence or by separate routes, not in compositions comprising more than one active ingredient. However, there are several published patent applications and approved patents teaching methods of treating respiratory disorders like asthma and chronic obstructive pulmonary disease (COPD) and pharmacologic compositions of different biologic and chemical substances for this purpose, where the combinations offer overall advantages in the treatment of these conditions. See for instance EP 0416950B1 “Medicaments”, EP 0416951B1 “Medicaments comprising salmeterol and fluticasone”, EP 0613371B1 “New combination of formoterol and budesonid”, WO 98/15280 “New combination”, WO 00/48587 “Combinations of formoterol and fluticasone propionate for asthma”, WO 01/70198A1 “Stabilized dry powder formulations”, WO 01/78737A1 “Medical combinations comprising formoterol and budesonid”, WO 01/78745A1 “Medical combinations comprising formoterol and fluticasone propionate”, WO 02/28368A1 “New combination for the treatment of asthma”, WO 03/013547A1 “Pharmaceutical composition comprising salmeterol and budesonid for the treatment of respiratory disorders”. However, the quoted documents deal with aspects of formulating, processing, stabilizing and using mixtures of at least two ingredients. The chemical compositions and mixing ratios between active ingredients are generally focused upon, not methods of administration of such compositions or devices for that purpose. It is, however, difficult to mix dry medicament powders and optional excipients in a certain proportion consistently. The proportions in such a metered combined dose cannot be easily controlled, because the ratio of medicaments in an individual, combined dose depends significantly on the particle forces existing in each medicament powder, between particles of different medicaments and between medicament powders and dose packaging materials. Hence, actual variations in the ratio between active ingredients from combined dose to combined dose may be too large, causing serious problems if a potent ingredient is delivered in a higher or lower amount than expected.

Thus, there is room for improvements regarding methods of treating respiratory disorders using combined, consistently metered doses of different medicaments for simultaneous administration by inhalation.

### SUMMARY

The present invention discloses a method for the prophylaxis or treatment of a respiratory disorder in a mammalian host by inhalation of a metered dry powder combined dose of finely divided dry medication powders. At least one dry powder medicament is selected from a first group of bronchodilating medicaments and at least one dry powder medicament from a second group of anti-inflammatory medicaments. A metered dry powder medicinal combined dose comprising separately metered deposits of medicinally suitable quantities of each of the selected medicaments is prepared, in which the sum of the metered deposits constitutes the metered quantities of powder of the combined dose and the medicinal combined dose is introduced into an inhaler device for delivery of the medicinal combined dose during the course of a single inhalation by a user, such that the delivered medicinal combined dose is composed of a high proportion of mixed de-aggregated fine particles of the selected medicaments respectively, whereby an intended therapeutic or treating effect to the user is achieved.

Furthermore a pharmaceutical dry powder combined dose is disclosed. The dose being adapted for inhalation, for the prophylaxis or treatment of a respiratory disorder in a mammalian host. In the combined dose at least one medicament from a first group of bronchodilating medicaments and at least one medicament from a second group of anti-inflammatory medicaments are selected and the pharmaceutical dry powder combined dose is prepared comprising separate, metered deposits of a medicinally suitable quantity of the selected medicaments from the first and second groups of medicaments respectively, where the sum of the deposits constitute the metered quantity of powder in the pharmaceutical, combined dose being introduced to an inhaler adapted device.

The present method is set forth by the independent claim 1 and the dependent claims 2 to 9, and a pharmaceutical combined dose is set forth by the independent claim 10 and the dependent claims 11 to 17.

### BRIEF DESCRIPTION OF THE DRAWINGS

The invention, together with further objects and advantages thereof, may best be understood by referring to the following detailed description taken together with the accompanying drawings, in which:

FIG. 1 illustrates in top and side views a first embodiment of a combined dose comprising two medicament deposits in separate compartments onto a dose bed;

FIG. 2 illustrates in top and side views a second embodiment of a combined dose comprising three medicament deposits in separate compartments onto a dose bed;

FIG. 3 illustrates in top and side views a third embodiment of a combined dose comprising two parallel medicament deposits onto a dose bed;

FIG. 4 illustrates in top and side views a fourth embodiment of a combined dose comprising several medicament deposits and separating excipient deposits onto a dose bed;

FIG. 5 illustrates in top and side views a fifth embodiment of a combined dose comprising four medicament deposits and separating excipient deposits onto a dose bed;

FIG. 6 illustrates in top and side views a sixth embodiment of a combined dose comprising two parallel medicament deposits on top of one another onto a dose bed;

FIG. 7 illustrates in top and side views a seventh embodiment of a combined dose comprising two medicament deposits on top of one another onto a dose bed, but separated by a deposit of an excipient;

FIG. 8 illustrates in top and side views another embodiment of a combined dose comprising two medicaments separately deposited onto a dose bed;

FIG. 9 illustrates in top and side views yet another embodiment of a combined dose comprising two medicaments separately deposited onto a dose bed, but with some degree of overlap;

FIG. 10a illustrates in a sectional view an example of a combined dose comprising two medicament deposits on top of one another but separated by a deposit of an excipient onto a dose bed and adjacent to the combined dose a nozzle in a starting position before the combined dose is released;

FIG. 10b illustrates in a sectional view an example of a combined dose comprising two medicament deposits on top of one another but separated by a deposit of an excipient onto a dose bed and adjacent to the combined dose a nozzle in a relative motion sucking up the powder particles to be dispersed into the air stream;

### DETAILED DESCRIPTION

The present invention is based on a new method of forming combined doses comprising more than one medicament, and a new therapeutic method of treating respiratory diseases like asthma by delivering such combined doses by an inhalation route to a user of a dry powder inhaler (DPI). “Asthma” is used in this document as a generic term for the different respiratory disorders known in the field of medicine.

In the context of this application the word “medicament” is defined as a pharmacological substance, which comprises at least one chemically or biologically active agent. Further, a medicament may exist in a pure form of one or more pure active agents, or a medicament may be a compound comprising one or more active agents, optionally formulated together with other substances, e.g. enhancers, carriers, diluents or exipients. From this point on, the term “excipient” is used to describe any chemical or biologic substance mixed in with a pure active agent for whatever purpose. In this document, only medicaments in dry powder form are discussed.

A “dose bed” is henceforth defined as a member capable of harboring a metered combined dose of one or more dry powders, where the combined dose is intended for delivery to a user of a DPI in a single inhalation performed by the user. In the present invention a combined dose for treating asthma comprises metered deposits of at least two medicaments. The dose bed may be divided in several areas or incorporate two or more compartments intended for deposits of dry powders. In a preferred embodiment the combined dose is packaged for a continuous, prolonged delivery, i.e. the delivery period is in a range 0.01 to 6 s, usually in a range 0.1 to 2 seconds, delivery taking place sometime during the course of an inhalation as controlled by a purposefully designed DPI. Advantageously, such a DPI adopts an Air-razor method of gradual aerosolizaton of the combined dose by introducing a relative motion between an air-sucking nozzle and the powder dose. Advantages of a prolonged delivery of a dose for inhalation are disclosed in our U.S. Pat. No. 6,571,793 B1 (WO 02/24264 A1), which is hereby incorporated in this document in its entirety as a reference.

A preferred embodiment of a metered combined dose uses a dose bed split up in at least two separate compartments, where each compartment is intended for a metered deposition of a particular asthma medicament. Each compartment containing a metered amount of a specified medicament powder may then be sealed, e.g. by foiling, such that the different medicaments in the different compartments of the dose bed cannot interact in any way and cannot be contaminated by foreign substances or moisture. Alternatively, a common foil encloses all compartments, but sealing between compartments may be excluded if individual sealing is not a requirement. A dose carrier is normally engaged to carry at least one dose bed loaded with a combined dose, whereby the dose carrier may be inserted into a DPI for administering a combined dose or doses sequentially to a user in need of treatment. A suitable carrier of combined doses is disclosed in our Swedish patent publication SE 0517 806 C2 WO 01/34233 A1), which is hereby incorporated in this document in its entirety as a reference. However, a dose bed may be designed to act as a carrier, intended for direct insertion into a DPI. A suitable DPI for a continuous dose delivery is disclosed in our U.S. Pat. No. 6,422,236 B1, which is hereby incorporated in this document in its entirety as a reference.

If complete physical separation of the deposits of the different medicaments making up the combined dose is not required, but some degree of overlap or mixing is acceptable from a physical, chemical and medical point of view, then other methods of separating the deposits may be implemented. Depending on what degree of mixing is permitted, different ways of separating deposits must be adopted. For example, in one embodiment, the different medicaments may be deposited in parallel strings onto the dose bed. The dose bed may use separate indentations where powder should be deposited, but flat target areas for deposits in a single plane on the dose bed are equal possible. In another embodiment the different medicaments are deposited sequentially dot-wise or string-wise onto different target areas of the dose bed. Yet another way of depositing the medicaments would be on top of one another, in layer by layer, such that each medicament is deposited on top of the previously deposited one. If necessary, to stop chemical or biological interaction or decomposition caused by, for example, adjacent medicament powders being incompatible, an isolating layer of a biologically acceptable, inert substance like carbohydrates, e.g. glucose or lactose, may be deposited between adjacent layers of medicaments. A similar method of separation may also be used to positively separate adjacent dots or strings of medicaments, by depositing an inert substance between adjacent dots or strings of different medicament deposits onto the dose bed. When the combined dose has been completely formed it is usually sealed from ingress of dirt and moisture by a foil covering the entire dose bed. A method of depositing microgram and milligram quantities of dry powders using electric field technology is disclosed in our U.S. patent application No. 2003/0012865 A1, which is hereby incorporated in this document in its entirety as a reference.

Forming a combined dose comprising at least two medicaments in separate dry powder formulations may be done in different ways, known in prior art The invention discloses that the components of the combined dose, i.e. the at least two medicaments, need not be med or processed together prior to dose forming and, indeed, should normally be kept separated during dose forming as well as after the combined dose is formed and sealed. The medicaments of the combined dose are thus kept separated on the dose bed by a suitable method, as described in the foregoing, until the combined dose is about to be delivered by an inhalation route to a user.

Methods of dose forming include conventional mass or volumetric metering and devices and machine equipment well known to the pharmaceutical industry for filling blister packs, for example. See European Patent No. EP 0319131B1 and U.S. Pat. No. 5,187,921 for examples of prior art in volumetric and/or mass methods and devices for producing doses of medicaments in powder form. Electrostatic forming methods may also be used, for example as disclosed in U.S. Pat. Nos. 6,007,630 and 5,699,649. Any method capable of producing metered microgram and milligram quantities of dry powder medicaments may be used, even completely different methods may be applied to suit the different medicaments selected to be part of the combined doses to be produced. Total mass in a combined dose according to the present invention is typically in a range from 50 μg to 50 mg. Regardless of which forming and fining method being used for a particular medicament, it is important during dose forming to make sure that selected medicaments are individually metered and deposited onto their respective target areas or compartments of the dose bed. The target areas or compartments of the dose bed, which aggregate to hold a combined dose, may be of a same size or different sizes. The shape of compartments is governed by physical constraints defined by the type of dose bed used. As an example, a preferred type of dose bed is an elongated strip of a biologically acceptable, inert material, e.g. plastic or metal, between 5 and 50 mm long and between 2 and 10 mm wide. The strip is further divided in separate target areas or compartments arranged along the length of the elongated strip. The dose bed or, if necessary each compartment, receives an individual seal, for instance in the form of a foil, in a step immediately subsequent to the dose forming.

An advantage of the present invention is that a potentially interesting asthma medicament may be individually selected on merits of its own for inclusion in a combined dose, in disregard of whether or not it is chemically or biologically compatible with other potentially interesting asthma medicaments. The combined dose may be designed to include medicaments, which have proven medical effects of different, desirable kinds, even though the selected medicaments may be chemically or biologically incompatible or unstable in the form of a mixture. Thus, the regulatory process before introducing combined doses of different asthma medicaments on the market may be drastically simplified. Yet another advantage of the invention is the possibility of using pure, more or less potent medical agents as selected medicaments of the combined dose, without any included excipients.

For example, in the treatment today of mild and severe asthma and other respiratory disorders such as rhinitis and chronic obstructive pulmonary disease (COPD) a bronchodilating compound for fast relief of symptoms is often used together with an anti-inflammatory steroid to control an airway inflammation, which is at the root of the disorder. In managing asthma three types of medications are typically used in therapy: Control medications, Prevention medications and Rescue medications.

Control medications comprise corticosteroids, e.g. fluticasone, non-steroidal anti-inflammatory drugs, e.g. sodium cromoglicate, and theophylline. Control medicines decrease or prevent the inflammation or redness and swelling in the airways and are generally called anti-inflammatory medications. They are first line treatment for long-term control of persistent (mild persistent, moderate persistent, or severe persistent) levels of asthma. There are long-acting and short-acting medicaments in this group, a long-acting drug is typically administered once a day, whereas a short-acting has to be administered more than once a day.

Prevention medications are different from control medications in that they are not anti-inflammatory medicines; they do not work by decreasing inflammation in the airways. Leukotriene modifiers, one type of prevention medicine, prevent asthma attacks by blocking the effects of leukotriene mediators, a potent active substance created as the result of an allergic response. Leukotriene modifiers have both a control and a prevent activity. Long-acting beta2-agonists, e.g. formoterol, act for at least 12 hours and work by relaxing the smooth muscles around the airways to prevent bronchospasm.

Rescue medications have fast on-set and start working right away and the effects are generally shorter-lasting than control or prevention medications. They are generally used in case of an acute asthma attack to quickly relieve symptoms such as shortness of breath, chest tightness, coughing, and wheezing. Short-acting beta2-agonists, e.g. albuterol and terbutaline, have a peak effect within 20 minutes and anticholinergics, e.g. ipratropium, provide fairly fast relief of symptoms, generally within one to two hours. Both types are commonly used drugs in this group. Long-acting beta2-agonist formoterol is also often used as rescue drug because of its fast on-set.

Illustrative examples, not limiting the scope of the invention, of suitable typical medicaments for treatment of asthma, which combine advantageously in single combined doses in accordance wit the present invention, are included non-exclusively in groups “A1”, “A2” and “B” below:

A1. Bronchodilating Substances (Including Pharmaceutically Acceptable Salts, Enantiomers, Racemates Hydrates, Solvates, or Mixtures Thereof)

Albuterol (also known as Salbutamol)

Bambuterol

Bitolterol

Broxaterol

Carbuterol

Clenbuterol

Etanterol

Fenoterol

Formoterol

Hexoprenaline

Imoxiterol

Isoetharine

Metaproterenol

Naminterol

Picumeterol

Pirbuterol

Procaterol

Rimiterol

Reproterol

Salmeterol

Terbutaline

Tulobuterol

A2. Anticolinergic, Bronchodilating Substances (Including Pharmaceutically Acceptable Salts, Enantiomers, Racemates Hydrates, Solvates, or Mixtures Thereof)

Ipratropium

Tiotropium

B. Anti-Inflammatory Substances (Including Pharmaceutically Acceptable Salts, Enantiomers, Racemates Hydrates, Solvates, or Mixtures Thereof)

Budesonide

Beclomethasone

Ciclesonide

Dexametasone

Fluisolide

Fluticasone

Mometasone

Triamcinolone

TABLE 1
Typical dosages of examples of medicaments in asthma therapy

| Medicament | Delivered dosage | Delivered dosage |
|---|---|---|
| active | range per combined | range per day for |
| agent | dose (μg) | adults (μg) |
| Formoterol | 1-50 | 1-100 |
| Budesonide | 20-1600 | 20-4800 |
| Fluticasone | 40-2000 | 50-5000 |
| Mometasone | 50-1500 | 50-4000 |

For example, a combined dose according to the invention comprises at least one medicament prepared from substances in group “A1” or “A2”, optionally mixed with one or more excipients, and at least one medicament prepared from substances from group “B”, optionally mixed with one or more excipients. A medicament prepared from a substance in one of the groups may be combined with another medicament prepared from another substance of the same group into the same combined dose, which by definition also includes medicaments from the other group.

A combined dose is intended for administration in a single inhalation, either irregularly when need arises, or more typically as part of a daily management regime. The number of combined doses administered regularly may vary considerably depending on the type of disorder, types of medicaments and their potencies. Optimal dosages of the respective active substances for prevention or treatment of respiratory disorders may be determined by those skilled in the art, and will vary with the selected compounds, their respective potency and the advancement of the disease condition. Furthermore, factors associated with the individual undergoing treatment determine correct dosages, such as age, weight, sex etc. Depending on what are correct dosages, the correct deposits by mass for the prepared medicaments may be calculated, such that metered deposits of each medicament to be included in the metered combined dose may be produced in a dose-forming step. In calculating a correct nominal deposit of mass for each medicament component the fine particle fraction, i.e. particles having a mass median aerodynamic diameter (MMAD) less than 5 μm, per component of the actual delivered dose must be taken into consideration. As discussed in the foregoing, the efficacy of inhalers differs considerably and it is thus important to include the expected efficacy of the chosen inhaler in the calculation of what is a suitable nominal mass deposit An example of a combined dose is one composed of the long-acting bronchodilating substance formoterol selected from group A1 and the anti-inflammatory substance budesonide, selected from group B. What constitutes suitable amounts of the two component medicaments and the respective optimal masses of formoterol and budesonide depend on the factors described in the foregoing, but generally the inhaled formoterol mass should be in a range from 2 to 40 μg and inhaled budesonide in a range from 20 to 600 μg. Another parameter to consider when forming the combined dose is the physical formulation of included medication powders. Formulation objectives may differ for the different medicament components of the combined dose. The particle aerodynamic size distribution by mass may be targeted differently for the different dose components in order to optimize the efficacy of each component in the treatment of asthma in a host user. For instance, the MMAD for a steroidal medicament component based on a substance from group B should be larger than that of a bronchodilating medicament component based on a substance from group A1. Whereas maximum penetration into the lungs is required of a bronchodilator, a minimum of systemic absorbance and maximum local deposition in the targeted area of the airways is required of the steroid.

Compared to prior art, more opportunities are opened up by the present invention in selecting medicaments based on existing compositions with proven medical effects, rather than first developing a mixture or formulation of different medicaments and then proving that the new combination is effective, stable and lacks serious side effects. The present invention makes it possible to define combined doses by using any combination of pure medicaments, ie. pure pharmacologic agents, and medicaments comprising excipients. A combined dose thus formed may be introduced into a dry powder inhaler (DPI) such that the medicament components making up the combined dose may be aerosolized and delivered in the inspiration air during the course of an inhalation through the DPI by a user.

The invention also offers interesting opportunities for combinations of new, more effective medicaments and combinations of new medicaments with existing, proven ones. Keeping the different medicaments separated according to the invention may reduce the investment in time and resource necessary for getting the combined medicaments approved by the relevant regulatory bodies and released to the respective markets. For instance, no added substance to stabilize the combined product will be needed and no testing to prove that the added substance is harmless needs be performed.

The present invention differs from prior art inhalers and related combined dose delivery methods by providing a combined dose comprising two or more separate asthma medicaments, more or less separately deposited onto a dose bed. The combined dose is therefore not a composition of asthma medicaments constituting a single physical entity, but rather two or more physical entities contained in a single combined dose for treatment of asthma. Inserted into a DPI, the combined dose will be aerosolized such that the entities of the combined dose, the medicaments, will be delivered mostly sequentially or optionally mostly simultaneously into the inspiration air during an inhalation by a user. Whether medicaments included in a combined dose are aerosolized mainly sequentially or mainly simultaneously depends party on the physical form of the combined dose, i.e. how the medicament deposits are interrelated and partly on what type of inhaler is used to administer the combined dose. It is obvious that an inhaler, which instantaneously subjects all powders of the combined dose to a jet-stream of air will aerosolize the aggregated deposits simultaneously and more or less mixed, whereas an inhaler subjecting the combined dose to a jet stream gradually, like a moving tornado attacking a corn field, thereby not attacking all of the combined dose instantly, may aerosolize the deposits of the combined dose gradually over time. An object of the invention is to offer better control of combined dose release and to facilitate a prolonging of the combined dose delivery in order to produce a high fine particle fraction (FPF) in the delivered, combined dose. Another object of the invention is to achieve a high ratio of delivered, combined dose relative metered, combined dose. Although it is possible to successfully apply the invention to prior art inhalers, they tend to deliver the combined dose in too short a time, resulting in a poor FPF figure and low efficacy. On the other hand, a gradual combined dose delivery is possible using a new inhaler design where a relative movement is introduced between the combined dose and a suction nozzle through which the inspiration airflow is channeled. This arrangement utilizes the inhalation effort of the user to aerosolize the combined dose gradually for a prolonged period, thus using the power of the suction more efficiently and eliminating in most cases a need for external power to aerosolize the combined dose.

A powder Air-razor method is advantageously used for aerosolizing the medicament powders in the combined dose, the Air-razor providing de-aggregation and dispersal into air of the finely divided medication powders Utilizing an effort of sucking air through a mouthpiece of an inhaler, said mouthpiece connected to a nozzle, the particles of the deposited medicament powders, made available to the nozzle, are gradually de-aggregated and dispersed into a stream of air entering the nozzle. The gradual de-aggregation and dispersal is produced by the high shearing forces of the streaming air and a relative motion introduced between the nozzle and the powders of the combined dose. In a preferred embodiment, the medicament powders are deposited onto a dose bed, such that the powder deposits occupy an area of similar or larger size than the area of the nozzle inlet. The nozzle is preferably positioned outside the area of deposits, not accessing the powder by the relative motion until the air stream into the nozzle, created by an applied suction, has passed a threshold flow velocity. Coincidental with the application of the suction or shortly afterwards the relative motion will begin such that the nozzle traverses the combined powder dose gradually. The high velocity air going into the nozzle inlet provides plenty of shearing stress and inertia energy as the flowing air hits the leading point of the border of the contour of the first medicament deposit. This powder Air-razor method, created by the shearing stress and inertia of the air stream, is so powerful that the particles in the particle aggregates in the powder adjacent to the inlet of the moving nozzle are released, de-aggregated to a very high degree as well as dispersed and subsequently entrained in the created air stream going through the nozzle. If the medicament deposits have been made in separate compartments of the dose bed and individually sealed, then obviously the compartments must be opened up first so that the nozzle can access the deposited powder in each compartment when suction is applied. Naturally, this is also true if the deposits share a common seal without an individual seal for each deposit. An arrangement for cutting foil is disclosed in our Swedish patent publication SE 517 227 C2 (WO 02/24266 A1), which is hereby incorporated in this document in its entirety as a reference. Depending on how the deposits are laid out on the dose bed, the nozzle will either suck up the deposits sequentially or in parallel or in some serial/parallel combination.

Thus, the quality of asthma dose delivery is dramatically improved compared to prior art performance, leading to important advances in delivering a majority of fine particles of the asthma medicaments of the combined dose to the intended target area or areas in the user's airways and lungs with very little loss of particles settling in the throat and tipper airways. Administering asthma medicament combinations according to the present invention has a very positive therapeutic effect from a medical, psychological and social point of view on a host in need of asthma treatment with a combination of at least two medicaments.

Detailed Descriptions of Drawings

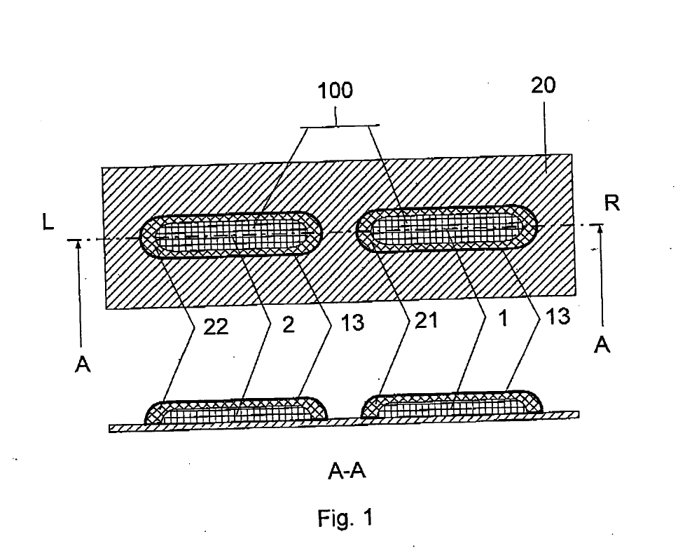

Referring to reference numbers 1-100 of the drawings wherein like numbers indicate like elements throughout the several views of ten different embodiments of a combined dose comprising at least two deposits of at least two medicaments onto a dose bed as illustrated in FIGS. 1-10 presented here as non-limiting examples.

FIG. 1 illustrates a combined dose 100 comprising two different medicament deposits, 1 and 2, in separate compartments 21 and 22 onto a dose bed 20, said compartments may be capsules or blisters or moldings in the dose bed. An individual seal 13 for each compartment guarantees that the medicaments cannot be contaminated by foreign matter or by one another. The illustrated deposits are intended for a sequential delivery taking place during an inhalation.

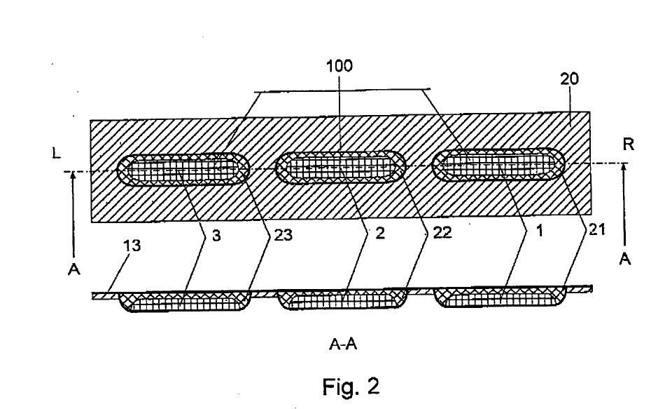

FIG. 2 illustrates a combined dose 100 comprising three different medicament deposits, 1, 2 and 3 in separate compartments 21, 22 and 23 similar to FIG. 1, but arranged underneath the dose bed 20. Besides a different arrangement of compartments on the dose bed 20 and the respective seals 13, the main difference between FIG. 1 and FIG. 2 is that deposit 3 may consist of a different medicament from deposits 1 and 2 or it may consist of either the medicament of deposit 1 or 2. It is thus possible not only to administer more than one medicament, but also to compose combined doses of e.g. two medicaments with a very high ratio of mass between them. The illustrated deposits are intended for a sequential delivery taking place during an inhalation.

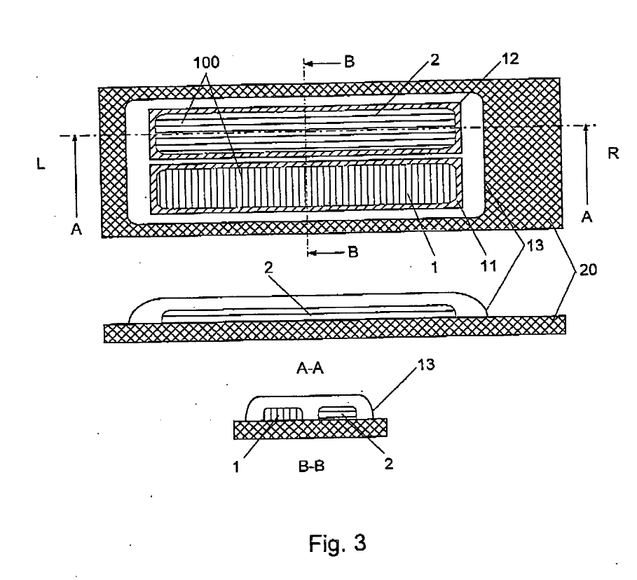

FIG. 3 illustrates a combined dose 100 comprising two different medicament deposits, 1 and 2, laid out in parallel strips onto separate target areas 11 and 12 respectively onto the dose bed 20. A common protective foil 13 protects the medicaments of the combined dose from being contaminated by foreign matters. The illustrated deposits are intended for a fully simultaneous delivery of the two medicaments taking place during an inhalation.

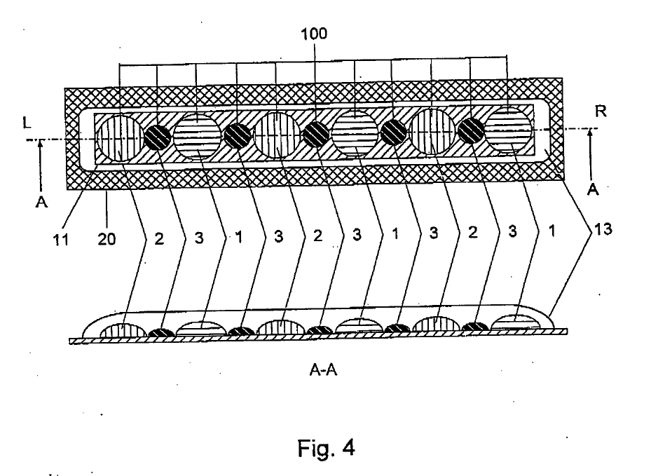

FIG. 4 illustrates a combined dose 100 comprising two different medicaments, 1 and 2, each comprising several deposits separated by deposits of an inert excipient 3. The deposits are laid out in a string of spots onto a target area 11 on a dose bed 20. The deposits share a common seal 13. The combined dose is intended for a sequential delivery of incorporated medicament spots, said delivery taking place during an inhalation. The excipient deposits help to minimize unintentional mixing of the medicaments. If some nixing of medicaments can be accepted, then the excipient may be left out altogether. Combined doses composed of spot deposits may of course comprise more medicaments than two. The mass ratio between medicaments may be easily set by controlling the ratio between the number of spots per medicament in combination with the size of the respective spots in terms of deposited mass. Naturally the spots need not necessarily be circular in shape, they may take an elongated or elliptical form, depending on which types of combined dose forming methods are used.

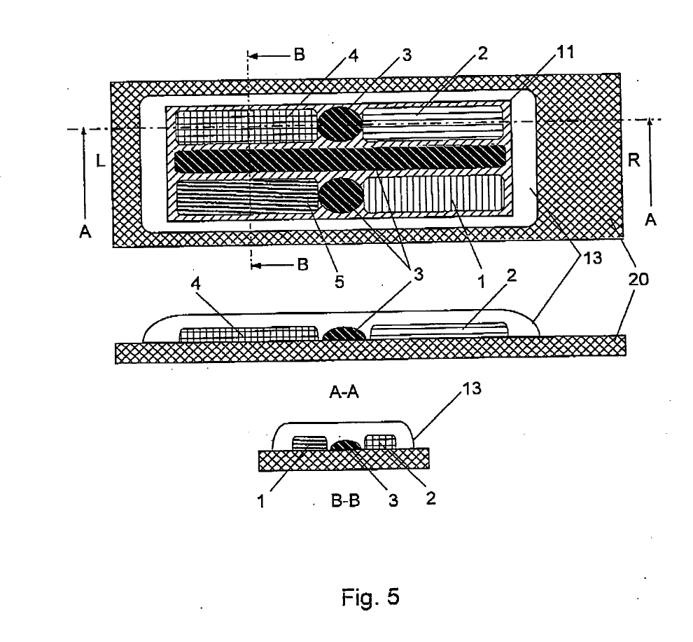

FIG. 5 illustrates a combined dose 100 comprising deposits of four different medicaments, 1, 2, 4 and 5, separated by deposits of an inert excipient 3. The deposits are laid out in two parallel groups of two medicaments per group in lined strips onto a common target area 11 on a dose bed 20. The deposits share a common seal 13. The excipient deposits help to minimize unintentional interaction of the medicaments. The combined dose is intended for a combined parallel/simultaneous and sequential delivery of incorporated medicaments, said delivery taking place during an inhalation.

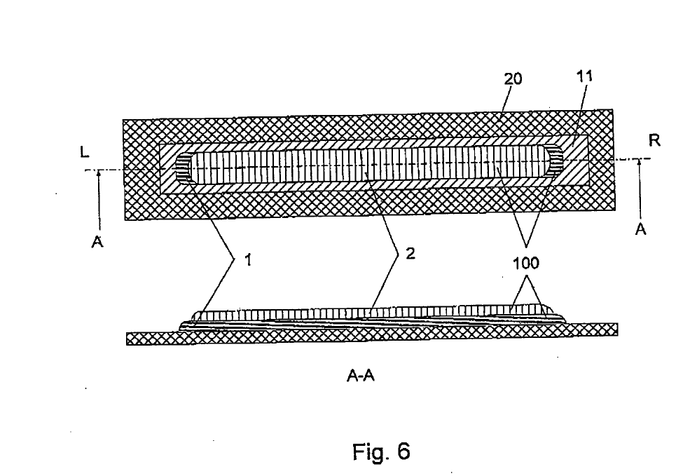

FIG. 6 illustrates a combined dose 100 comprising two different medicaments, 1 and 2, each comprising a strip of deposited powder, medicament 1 deposited onto a target area 11 of a dose bed 20 and medicament 2 deposited on top of the deposit of medicament 1. This method of combined dose forming is an alternative to the ones previously disclosed and may be used when a certain level of interaction of the medicaments can be tolerated.

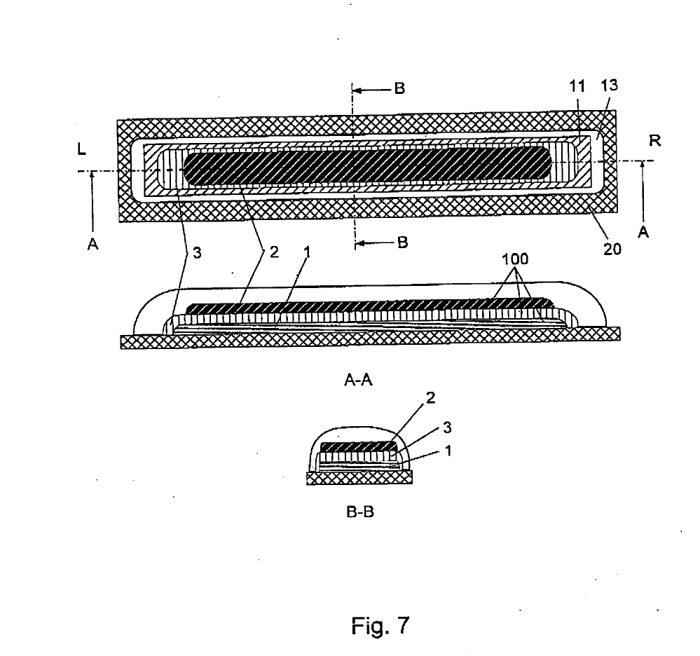

FIG. 7 illustrates a combined dose 100 comprising two different medicaments, 1 and 2, and an excipient 3, each comprising a strip of deposited powder. Medicament 1 is deposited onto a target area 11 of a dose bed 20 and excipient 3 is deposited onto medicament 2 to insulate medicament 1 from a deposit of medicament 2 on top of the deposits of medicament 1 and excipient 3. This way of forming combined doses is not restricted to include only two medicaments, but several medicaments may be deposited on top of one another, if necessary with an insulating deposit of excipient between layers.

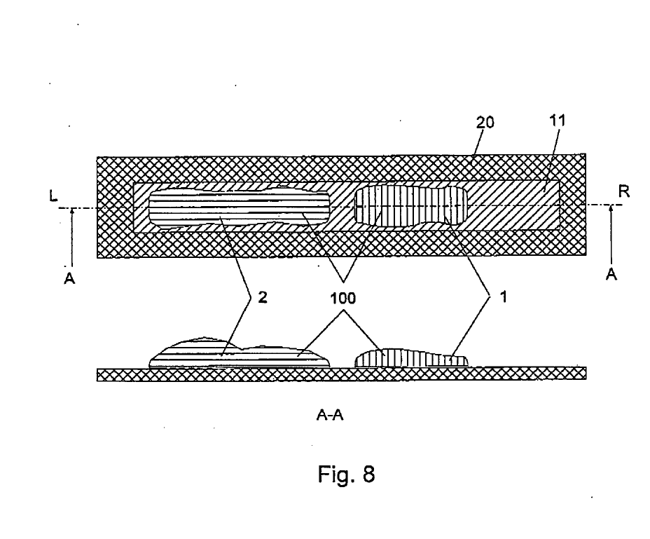

FIG. 8 illustrates a combined dose 100 comprising two different medicament deposits, 1 and 2, of somewhat irregular shapes but separately laid out onto a common target area 11 of the dose bed 20. The illustrated deposits are intended for a sequential delivery of the two medicaments taking place during an inhalation.

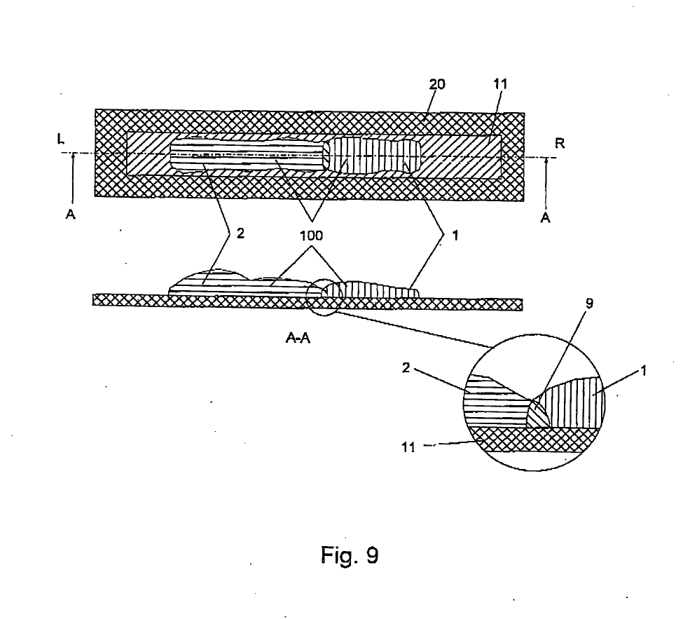

FIG. 9 illustrates a combined dose 100 comprising two different medicament deposits, 1 and 2, of somewhat irregular shapes but generally separately laid out onto a common target area 11 of the dose bed 20. The illustrated deposits overlap slightly, resulting in a arbitrary mixture 9. The deposits are intended for a mostly sequential delivery of the two medicaments taking place during an inhalation.

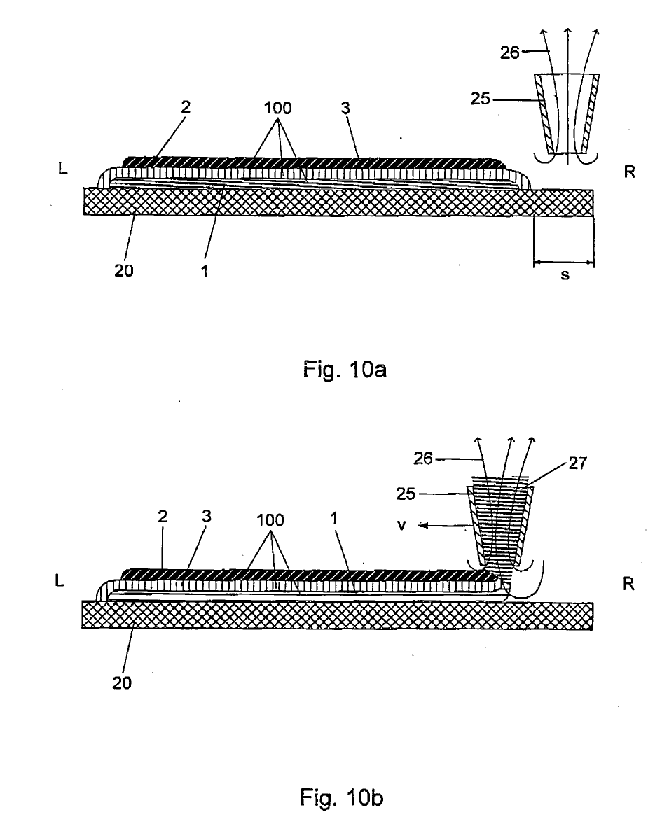

FIGS. 10a and 10b illustrate a delivery of a combined dose 100 comprising two different medicaments, 1 and 2, and an excipient 3, each comprising a strip of powder sequentially deposited in three different layers. A nozzle 25 with an established flow of air 26 going into it is put in a relative motion, parallel to the dose bed 20, such that the nozzle passes over the combined dose beginning at the right side R and ending at the left side L of the dose bed. This Air-razor method results in a simultaneous, gradual delivery of medicaments 1 and 2 together with the excipient 3. The powders of the deposits are mixed into an aerosol 27 by the air flowing into the nozzle leading to simultaneous delivery of the two medicaments and the excipient. This Air-razor method may be applied to all embodiments of the present invention and results in a simultaneous or sequential or a combined simultaneous/sequential delivery of all included medicaments and optional excipients.

1. A method for the prophylaxis or treatment of a respiratory disorder in a mammalian host by inhalation of a metered dry powder combined dose of finely divided dry medication powders, characterized by the steps of
selecting at least one dry powder medicament from a first group of bronchodilating medicaments and at least one dry powder medicament from a second group of anti-inflammatory medicaments;
preparing a metered dry powder medicinal combined dose comprising separately metered deposits of medicinally suitable quantities of each of the selected medicaments, where the sum of the metered deposits constitutes the metered quantity of powder of a medicinal combined dose;
introducing the medicinal combined dose into an inhaler device for delivery of the medicinal combined dose during the course of a single inhalation by a user, such that the delivered medicinal combined dose is composed of a high proportion of mixed de-aggregated fine particles of the selected medicaments respectively, whereby an intended therapeutic or treating effect to the user is achieved.

2. The method according to claim 1, characterized by the further step of
using formoterol or a pharmaceutically acceptable salt, enantiomer, racemate, hydrate, solvate, or mixtures thereof from the first group of bronchodilating medicaments as a first medicament and budesonide or a pharmaceutically acceptable salt, enantiomer, racemate, hydrate, solvate, or mixtures thereof from the second group of anti-inflammatory medicaments as a second medicament.

3. The method according to claim 1, characterized by the flitter step of
using formoterol or a pharmaceutically acceptable salt, enantiomer, racemate, hydrate, solvate, or mixtures thereof from the first group of bronchodilating medicaments as a first medicament and fluticasone or a pharmaceutically acceptable salt, enantiomer, racemate, hydrate, solvate, or mixtures thereof from the second group of anti-inflammatory medicaments as a second medicament.

4. The method according to claim 1, characterized by the further step of
using formoterol or a pharmaceutically acceptable salt, enantiomer, racemate, hydrate, solvate, or mixtures thereof from the first group of bronchodilating medicaments as a first medicament and mometasone or a pharmaceutically acceptable salt, enantiomer, racemate, hydrate, solvate, or mixtures thereof from the second group of antiinflammatory medicaments as a second medicament.

5. The method according to claim 1, characterized by the further step of
using formoterol or a pharmaceutically acceptable salt, enantiomer, racemate, hydrate, solvate, or mixtures thereof from the first group of bronchodilating medicaments as a first medicament and ciclesonide or a pharmaceutically acceptable salt, enantiomer, racemate, hydrate, solvate, or mixtures thereof from the second group of antiinflammatory medicaments as a second medicament.

6. The method according to claim 1, characterized by the further step of
using one or more of the substances Albuterol (also known as Salbutamol), Bambuterol, Bitolterol, Broxaterol, Carbuterol, Clenbuterol, Etanterol, Fenoterol, Formoterol, Hexoprenaline, Imoxiterol, Isoetharine, Metaproterenol, Naminterol, Picumeterol, Pirbuterol, Procaterol, Rimiterol, Reproterol, Salmeterol, Terbutaline, Tiotropium and Tulobuterol or pharmaceutically acceptable salts, enantiomers, racemates, hydrates, solvates, or mixtures thereof belonging to the first group of bronchodilating medicaments as a first medicament and one or more of the substances Budesonide, Beclomethasone, Ciclesonide, Dexametasone, Flunisolide, Fluticasone, Ipratropium, Mometasone and Triamcinolone or pharmaceutically acceptable sats, enantiomers, racemates, hydrates, solvates, or mixtures thereof belonging to the second group of anti-inflammatory medicaments as a second medicament.

7. The method according to claim 1, characterized by the further step of
preparing the dry powder medicinal combined dose to a total mass in a range from 10 μg to 50 mg.

8. The method according to claim 1, characterized by the further step of
separating the deposits of the included medicaments from each other onto a dose bed, such that the medicaments cannot detrimentally mix with each other after forming of the combined dose.

9. The method according to claim 1, characterized by the further step of
selecting a continuous dry powder inhaler (DPI) designed for a prolonged delivery of the medicinal combined dose to a user inhaling once through the DPI.

10. A pharmaceutical dry powder combined dose, adapted for inhalation, for the prophylaxis or treatment of a respiratory disorder in a mammalian host characterized in that
at least one medicament from a first group of bronchodilating medicaments and at least one medicament from a second group of anti-inflammatory medicaments are selected;
the pharmaceutical dry powder combined dose is prepared comprising separate, metered deposits of a medicinally suitable quantity of the selected medicaments from the first and second groups of medicaments respectively, where the sum of the deposits constitute the metered quantity of powder in the pharmaceutical, combined dose;
the pharmaceutical dry powder combined dose is introduced into an inhaler device for a user initiated delivery of the pharmaceutical dry powder combined dose, whereby the first and second medicaments of the combined dose are delivered to the host user during the course of a single inhalation;
the combined therapeutical effect of the inhaled medicinal dosage comprising two selected medicaments is medically, psychologically or socially beneficial to the host user in need of such combined treatment.

11. The pharmaceutical dry powder combined dose according to claim 10, characterized in that
formoterol or a pharmaceutically acceptable salt, enantiomer, racemate, hydrate, solvate, or mixtures thereof is selected from the first group of bronchodilating medicaments as a first medicament and budesonide or a pharmaceutically acceptable salt, enantiomer, racemate, hydrate, solvate, or mixtures thereof is selected from the second group of anti-inflammatory medicaments as a second medicament.

12. The pharmaceutical dry powder combined dose according to claim 10, characterized in that
formoterol or a pharmaceutically acceptable salt, enantiomer, racemate, hydrate, solvate, or mixtures thereof is selected from the first group of bronchodilating medicaments as a first medicament and fluticasone or a pharmaceutically acceptable salt, enantiomer, racemate, hydrate, solvate, or mixtures thereof is selected from the second group of anti-inflammatory medicaments as a second medicament.

13. The pharmaceutical dry powder combined dose according to claim 10, characterized in that
formoterol or a pharmaceutically acceptable salt, enantiomer, racemate, hydrate, solvate, or mixtures thereof is selected from the first group of bronchodilating medicaments as a first medicament and mometasone or a pharmaceutically acceptable salt, enantiomer, racemate, hydrate, solvate, or mixtures thereof is selected from the second group of anti-inflammatory medicaments as a second medicament.

14. The pharmaceutical dry powder combined dose according to claim 10, characterized in that
formoterol or a pharmaceutically acceptable salt, enantiomer, racemate, hydrate, solvate, or mixtures thereof is selected from the first group of bronchodilating medicaments as a first medicament and ciclesonide or a pharmaceutically acceptable salt, enantiomer, racemate, hydrate, solvate, or mixtures thereof is selected from the second group of anti-inflammatory medicaments as a second medicament.

15. The pharmaceutical dry powder combined dose according to claim 10, characterized in that
one or more of the substances Albuterol (also known as Salbutamol), Bambuterol, Bitolterol, Broxaterol, Carbuterol, Clenbuterol, Etanterol, Fenoterol, Formoterol, Hexoprenaline, Imoxiterol, Isoetharine, Metaproterenol, Naminterol, Picumeterol, Pirbuterol, Procaterol, Rimiterol, Reproterol, Salmeterol, Terbutaline, Tiotropium and Tulobuterol or pharmaceutically acceptable salts, enantiomers, racemates hydrates, solvates, or mixtures thereof belonging to the first group of bronchodilating medicaments may be used as a first medicament and one or more of the substances Budesonide, Beclomethasone, Ciclesonide, Dexametasone, Flunisolide, Fluticasone, Ipratropium, Mometasone and Triamcinolone or pharmaceutically acceptable salts, enantiomers, racemates hydrates, solvates, or mixture thereof belonging to the second group of antiinflammatory medicaments may be used as a second medicament.

16. The pharmaceutical dry powder combined dose according to claim 10, characterized in that
the combined dose is prepared to a total mass in a range from 10 μg to 50 mg.

17. The pharmaceutical dry powder combined dose according to claim 10, characterized in that
the deposits of the included medicaments are suitably separated from each other onto a dose bed, such that the medicaments cannot detrimentally mix with each other after forming of the combined dose.
